(19) United States
(12) Patent Application Publication
(10) Pub. No.: US 2018/0222831 A1
(43) Pub. Date: Aug. 9, 2018

(54) METHOD FOR THE PRODUCTION OF AT LEAST ONE DERIVATE OF A CARBOXYLIC ACID

(71) Applicant: White Dog Labs, Inc., New Castle, DE (US)

(72) Inventors: Michael E. WRIGHT, Ridgecrest, CA (US); Aharon M. EYAL, Jerusalem (IL)

(73) Assignee: White Dog Labs, Inc., New Castle, DE (US)

(21) Appl. No.: 15/750,054

(22) PCT Filed: Aug. 5, 2016
(86) PCT No.: PCT/US2016/045858

§ 371 Date: Feb. 2, 2018

Related U.S. Application Data
(60) Provisional application No. 62/201,344, filed on Aug. 5, 2015.

(51) Int. Cl. C07C 29/149; C07C 67/08; C12P 7/40

(52) U.S. Cl. CPC C07C 29/149; C12P 7/40; C07C 67/08

(57) ABSTRACT

A method of producing at least one derivate of a carboxylic acid. The method includes reacting a carboxylic acid with an auxiliary alkanol on a first catalyst to form a produced ester. A fraction of the produced ester is optionally separated to form a first separated ester. At least a fraction of said produced ester is reacted with hydrogen on a second catalyst to produce a mixture of product alkanol, auxiliary alkanol and optionally residual ester. The product alkanol is separated from the auxiliary alkanol in the mixture to form separated product alkanol, separated auxiliary alkanol, and optionally a second separated ester. The separated auxiliary alkanol is recycled to the reaction. Methods and catalysts for converting alcohols and acids to hydrocarbon jet and diesel fuels are also described.

### CROSS REFERENCE TO RELATED APPLICATIONS

The instant application claims priority to U.S. Provisional Application No. 62/201,344, filed Aug. 5, 2015, the disclosure of which is incorporated by reference herein in its entirety.

### TECHNICAL FIELD

The field of art to which the invention generally pertains is methods for the isolation of organic acids and production of derivates thereof.

### BACKGROUND

n-Butanol provides a linear carbon chain that can be dehydrated to 1-butene, an alpha-olefin, with high selectivity. On the other hand, n-butanol can also be readily converted to a mixture of 1-butene and 2-butenes (cis & trans) using a solid acid catalyst in a heated fixed-bed reactor. n-Butanol is also used in the preparation of two commercially relevant esters: butyl acetate and butyl methacrylate. Thus, n-butanol is not only a versatile synthetic intermediate but also has a direct use in the chemicals market.

α-Olefins are useful intermediates in preparing dienes, including 1,3-butadiene, diesel and jet/turbine fuels, lubricants, and polymers. 1-Butene is an especially useful precursor to 1,3-butadiene, which is used in preparing synthetic rubber and other advantageous polymeric elastomers. α-Olefins are also useful in preparing poly-α-olefins (PAOs) and copolymers with ethylene to form low-density plastics and with styrene to form elastomeric materials. Renewable α-olefin is useful in preparing the corresponding renewable products, including renewable fuels, polymers, elastomers, lubricants, PAOs, and other chemical intermediates.

Commercial production of bio-1-butanol has a rich history of successful large-scale production since the discovery by Louis Pasteur in the 1860s of bacteria that can ferment sugars to 1-butanol. Since Pasteur's initial discovery of the acetone-butanol-ethanol (ABE) process, many advances have been made in the fermentation process to optimize bio-1-butanol production and to reduce ethanol and acetone co-production. Most notable are the successful efforts using Clostridium bacteria in commercial plants developed by Weizmann at the turn of the 20^{th} century. With recent advances in fermentation and molecular biology strategies, bio-1-butanol can be produced that is cost competitive to and even less expensive than current petroleum-derived 1-butanol. One current drawback to the direct production of n-butanol, iso-butanol, and 1,4-butanediol is the loss of carbon (˜33%) during the fermentation process.

On the other hand, the production of butyric acid from methanol feedstocks or sugar and methanol feedstocks can be accomplished with a high retention of carbon. Other than consumption in creating biomass, butyric acid fermentation can be performed so that it does not emit carbon dioxide, and in fact, when using only methanol feedstock or feedstocks with a high ratio of methanol to sugar, the fermentation requires exogenous carbon dioxide and incorporates that carbon into the butyric acid product.

Hence, a method enabling high yield conversion of fermentation-produced carboxylic acid, e.g. butyric acid, into a product alkanol, e.g. butanol, would enable high yield conversion of carbon sources into alkanols. Such conversion of carboxylic acid into product alkanol should be cost effective and environmental friendly.

### SUMMARY OF THE INVENTION

Provided is a method for the production of at least one derivate of a carboxylic acid RCOOH, comprising (i) reacting in a reactor a carboxylic acid RCOOH with an auxiliary alkanol R′OH on a first catalyst to form a produced ester RCOOR′; (ii) optionally, separating a fraction of said produced ester RCOOR′ to form a first separated ester RCOOR′; (iii) reacting in said reactor at least a fraction of said produced ester RCOOR′ with hydrogen on a second catalyst to produce a mixture comprising a product alkanol RCH₂OH and said auxiliary alkanol R′OH and optionally residual ester RCOOR′; (iv) separating said product alkanol RCH₂OH and said auxiliary alkanol R′OH from said mixture to form separated product alkanol RCH₂OH, separated auxiliary alkanol R′OH and optionally a second separated ester RCOOR′; and (v) recycling said separated auxiliary alkanol R′OH to said reactor; wherein R and R′ denote alkyl and/or aryl groups, and steps (i) and (iii) are conducted in said reactor concurrently.

According to an embodiment, R in said carboxylic acid RCOOH comprises 1 to 30 carbon atoms. According to an embodiment, R′ in said auxiliary alkanol R′OH comprises 1 to 30 carbon atoms.

According to an embodiment, said carboxylic acid RCOOH is butyric acid and said product alkanol RCH₂OH is butanol.

According to an embodiment, said method further comprises producing a carboxylic acid RCOOH and obtaining a solution comprising said carboxylic acid RCOOH and said auxiliary alkanol R′OH, wherein said separated auxiliary alkanol R′OH is present during at least part of said carboxylic acid RCOOH-producing step.

According to an embodiment, said method further comprises producing a carboxylic acid RCOOH and obtaining a solution comprising said carboxylic acid RCOOH and at least one separated ester RCOOR′, wherein at least one of said first separated ester RCOOR′ and said second separated ester RCOOR′ are present during at least part of said carboxylic acid RCOOH-producing step.

According to an embodiment, said method further comprises producing carboxylic acid RCOOH in a fermentation process to form a fermentation liquor comprising said carboxylic acid RCOOH and separating said carboxylic acid RCOOH from said fermentation liquor.

According to an embodiment, said separating from said fermentation liquor comprises contacting said fermentation liquor with an extractant comprising separated auxiliary alkanol R′OH to form an extract solution comprising auxiliary alkanol R′OH and carboxylic acid RCOOH, and wherein the method further comprises adding said extract solution to said reactor.

According to an embodiment, said extractant further comprises a modifier and said extract solution comprises an auxiliary alkanol R′OH, a carboxylic acid RCOOH and said modifier, and the method further comprises removing at least a fraction of said modifier from the extract solution prior to or concurrently with said adding to said reactor. According to an embodiment, said modifier comprises a saturated or an unsaturated hydrocarbon containing from 3 to 30 carbon atoms.

According to an embodiment, said separating from said fermentation liquor comprises contacting said fermentation liquor with an extractant comprising a first and/or second separated ester RCOOR′ to form an extract solution comprising said first and/or second separated ester RCOOR′ and said carboxylic acid RCOOH, and wherein the method further comprises adding said extract solution to said reactor.

According to an embodiment, said extractant further comprises a modifier and said extract solution comprises said ester RCOOR′, said carboxylic acid RCOOH and said modifier, and the method further comprises removing at least a fraction of said modifier from the extract solution prior to or concurrently with said adding of said extract solution to said reactor. According to an embodiment, said modifier comprises a saturated or an unsaturated hydrocarbon containing from 3 to 30 carbon atoms.

According to an embodiment, the pH of said fermentation liquor is >5.5 and said separating further comprises acidulating with at least one of a mineral acid and an acidic cation exchanger.

According to an embodiment, said method comprises providing a fermentation liquor comprising a carbon source, culturing in said liquor a carboxylic acid RCOOH-producing organism and adding a basic compound for pH control. According to an embodiment, said basic compound is calcium hydroxide and/or calcium carbonate and said mineral acid is sulfuric acid.

According to an embodiment, said carbon source is selected from the group consisting of sugars, glycerol, methanol, CO, CO₂, syngas, and combinations thereof.

According to an embodiment, said organism is one or more of: a member of the phylum Firmicutes, a member of the class Clostridia, a member of the genus Eubacterium, a Eubacterium limosum, and a Clostridium selected from Clostridium butyricum, Clostridium acetobutylicum, Clostridium saccharoperbutylacetonicum, Clostridium beijerickii, Clostridium saccharobutylicum, Clostridium pasteurianum, Clostridium kluyveri, Clostridium carboxidovorans, Clostridium phytofermentens, Clostridium thermocellum, Clostridium cellulolyticum, Clostridium cellulovorans, Clostridium clariflavum, Clostridium ljungdahlii, Clostridium acidurici, Clostridium tyrobutyricum, and Clostridium autoethanogenum.

According to an embodiment, said first catalyst comprises a polymeric material containing Lewis and/or Bronsted acid sites.

According to an embodiment, said second catalyst comprises aluminum oxide containing from 10 to 40 weight percent copper. According to an embodiment, said second catalyst is silanized

According to an embodiment, said second catalyst comprises copper-containing aluminum oxide having a silanized surface. According to an embodiment, said second catalyst comprises an inorganic oxide, a transition metal, and a silanized surface.

According to an embodiment, both said first catalyst and said second catalyst comprise an active moiety supported on a support and the active moiety of the first catalyst and the active moiety of the second catalyst are commonly supported on the same support.

According to an embodiment, said reactor is maintained at a temperature between about 50° C. and 300° C. According to an embodiment it is maintained at a pressure between 1 psig and 1000 psig.

According to an embodiment, said reactor further comprises water at a weight fraction between 0.1% and 20%.

According to an embodiment, the molar yield of converting carboxylic acid RCOOH into product alkanol RCH₂OH is greater than 90%.

According to an embodiment, the method further comprises dehydrating said separated product alkanol RCH₂OH on a third catalyst. According to an embodiment, said third catalyst comprises a silanized inorganic metal oxide. According to an embodiment, said catalyst is a silanized, surface-stable selective dehydration catalyst for internal alkene production, comprising (i) an inorganic support, and (ii) at least one silicon compound.

### BRIEF DESCRIPTION OF THE DRAWINGS

FIG. 1 demonstrates an embodiment for performing methods as described herein comprising the use of an extraction column.

### DETAILED DESCRIPTION

It is to be understood that the foregoing general description and the following detailed description are exemplary and explanatory only and are not to be viewed as being restrictive of the invention, as claimed. Further advantages of this invention will be apparent after a review of the following detailed description of the disclosed embodiments, which are illustrated schematically in the accompanying drawings and in the appended claims.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed

### Definitions

The terms “terminal alkene,” “α-olefin,” “terminal olefin,” and “1-olefin” are used interchangeably herein to refer to an alkene with a double bond between the terminal carbon (carbon at the end of a hydrocarbon chain).

A “1-alcohol” or “terminal alcohol” or “primary alcohol” refers to an alcohol with a hydroxyl group attached to a terminal and primary carbon (i.e., a —CH₂OH group).

Butanoic acid refers to butyric acid, also abbreviated as BA.

For α-olefins, alkenes, dienes, alcohols, diols, solvents, and all chemical reagents used in descriptions within this document, IUPAC and/or standard organic chemical nomenclature used and accepted by the American Chemical Society (ACS) takes priority and is used in a way to match common nomenclature for clarity in the disclosure.

“Biofuel,” “biolubricant,” or “bio-1-butene” refers to a fuel, a lubricant, or 1-butene, respectively, that is produced from at least one biologically-produced starting molecule or at least one starting molecule in which at least one carbon atom is derived from a biological material, for example, xylose. Also the direct biological conversion of carbon dioxide and/or carbon monoxide is considered a viable route to bio-carboxylic acids. In one embodiment, a biofuel, biolubricant, or bio-1-butene may be produced from a bio-carboxylic acid that is produced by a biological process as described herein. Each of the bio-products will have a unique carbon-13/carbon-12 ratio that is dependent upon the feedstock and chemical process(s) described herein.

“Chemoselectivity” or “chemical selectivity” refers to a specific chemical product being formed selectively over other potential products if not otherwise defined. For example, if butyric acid is hydrogenated to 90 mol-% n-butanol and 10 mol-% of butanal, then this reaction has a 90% chemical selectivity for n-butanol.

“Chemical Yield” and “Yield” are calculated by multiplying the conversion (per cent) by the chemical selectivity (percent). For example, if reaction A that has a 90% conversion with a 90% chemical selectivity for product X, then product X has an 81% chemical yield in reaction A.

“Incipient Wetness Impregnation” (IWP) is a technique that is used to modify a support material by treating the solid material with a solution containing at least one desired modifier (e.g., calcium acetate), sufficient to wet the entire solid surface and possibly fill the pore-volume. The solvent(s) used in the IWP process can be removed by application of heat, or reduced pressure, or both, leaving behind the modifiers dissolved in the IWP solution.

“Weight Hourly Space Velocity” (WHSV) is defined as the weight of chemical feedstock entering the reactor per hour divided by the weight of catalyst used. For example, if 10.0 kilograms per hour (kg/hr) of butyric acid is fed to a reactor containing 2.0 kg of catalyst, the WHSV is 5 hr⁻¹. The substrate feed rate, in units of WHSV, does not include any co-feeds, such as extraction solvent or water. For the above example then, a feed of 4 kg of butyric acid containing 6 kg of n-butanol (i.e. a net feed of 10 kg) to a 2.0 kg invention catalyst this would result in a WHSV of 2 hr⁻¹.

“butyrate” is the same as CH₃CH₂CH₂CO₂⁻

“pKa” is defined as the −log[Ka] for an acid where Ka=[H⁺][A⁻]/[HA]

Provided is a method for the production of at least one derivate of a carboxylic acid RCOOH, comprising (i) reacting in a reactor a carboxylic acid RCOOH with an auxiliary alkanol R′OH on a first catalyst to form a produced ester RCOOR′; (ii) optionally, separating a fraction of said produced ester RCOOR′ to form a first separated ester RCOOR′ (iii) reacting in said reactor at least a fraction of said produced ester RCOOR′ with hydrogen on a second catalyst to produce a mixture comprising a product alkanol RCH₂OH and said auxiliary alkanol R′OH and optionally residual ester RCOOR′; (iv) separating said product alkanol RCH₂OH and said auxiliary alkanol R′OH from said mixture to form separated product alkanol RCH₂OH, separated auxiliary alkanol R′OH and optionally a second separated ester RCOOR′; and (v) recycling said separated auxiliary alkanol R′OH to said reactor; wherein R and R′ denote alkyl and/or aryl groups, and steps (i) and (iii) are conducted in said reactor concurrently.

According to an embodiment, R in said carboxylic acid RCOOH comprises 1 to 30 carbon atoms, 1 to 20 carbon atoms, 1 to 10 carbon atoms or 2 to 8 carbon atoms.

According to an embodiment, R′ in said auxiliary alkanol R′OH comprises 1 to 30 carbon atoms, 1 to 25 carbon atoms, 1 to 20 carbon atoms or 2 to 18 carbon atoms.

According to an embodiment, R and/or R′ is an alkyl group. According to an embodiment, R and/or R′ is an aryl group. According to an embodiment, R and/or R′ carry no hetero-atoms, i.e. consist solely of carbon atoms and hydrogen atoms. According to an embodiment, R and/or R′ carry at least one hetero-atom. According to an embodiment, said hetero-atom is at least one of oxygen, nitrogen, sulfur and a halide atom. According to an embodiment, R and/or R′ carry at least one oxygen atom. According to an embodiment, R comprises a hydroxyl group and said carboxylic acid is hydroxycarboxylic acid. According to an embodiment, R comprises an acidic group and said carboxylic acid is a dicarboxylic acid. According to an embodiment, R comprises an acidic group and said carboxylic acid is an amino acid. According to an embodiment, R′ comprises an acidic group and said auxiliary alkanol is a hydroxycarboxylic acid.

According to an embodiment, said carboxylic acid is a hydroxycarboxylic acid comprising both a carboxylic acid group and a hydroxyl group. According to an embodiment, said carboxylic acid is a hydroxycarboxylic acid, a fraction of the hydroxycarboxylic acids serve as the carboxylic acid RCH₂COOH and another fraction serves as the auxiliary alcohol R′OH. Said embodiment is demonstrated using 3-hydroxypropionic acid (3HPA) CH₂(OH)CH₂COOH as an example. The ester formed on said reacting in a reactor on said first catalyst (step i) is a dimer of 3HPA, CH₂(OH)CH₂COOCH₂CH₂COOH, or an oligomer thereof. Hydrogenation of said dimer generates propylene glycol, the product alkanol and 3HPA, the auxiliary alkanol.

According to an embodiment, said carboxylic acid is a hydroxycarboxylic acid, a fraction of the hydroxycarboxylic acids serve as the carboxylic acid RCH₂COOH, another fraction serves as the auxiliary alcohol R′OH and said reactor further comprises a second auxiliary alkanol R″OH, where R″ denotes an alkyl and/or an aryl group. According to an embodiment, said carboxylic acid dimerizes or oligomerizes on said first catalyst and said second auxiliary alkanol R″OH acts there as a chain terminator, as demonstrated for the case where the hydroxycarboxylic acid is 3HP and R″OH is methanol, forming CH₂(OH)CH₂COOCH₂CH₂COOCH₃.

According to an embodiment, said carboxylic acid RCOOH is butyric acid. According to an embodiment, said product alkanol RCH₂OH is butanol. According to an embodiment, said carboxylic acid RCOOH is butyric acid and said product alkanol RCH₂OH is butanol.

According to an embodiment, said method further comprises producing a carboxylic acid RCOOH with said separated auxiliary alkanol R′OH and obtaining a solution comprising said carboxylic acid RCOOH and said auxiliary alkanol R′OH.

According to an embodiment, said method further comprises producing a carboxylic acid RCOOH with at least one of said first separated ester RCOOR′ and said second separated ester and obtaining a solution comprising said carboxylic acid RCOOH and at least one separated ester RCOOR′.

According to an embodiment, said method further comprises producing carboxylic acid RCOOH in a fermentation process to form a fermentation liquor comprising said carboxylic acid RCOOH.

According to an embodiment, said method comprises providing a fermentation medium comprising a carbon source and culturing in said medium a carboxylic acid RCOOH-producing organism to form a fermentation liquor comprising said acid. According to an embodiment, said carbon source is selected from the group consisting of sugars, glycerol, methanol, CO, CO2, syngas and combinations thereof. According to an embodiment, said fermentation medium comprises a nitrogen source. According to an embodiment, said fermentation medium comprises hydrogen. According to an embodiment, the concentration of the carboxylic acid RCOOH in said formed fermentation liquor is between 5 gram per liter (gr/L) and 100 gr/L.

According to an embodiment, said fermentation is mixotrophic. According to an embodiment, said organism is one or more of: a member of the phylum Firmicutes, a member of the class Clostridia, a member of the genus Eubacterium, a Eubacterium limosum, and a Clostridium selected from Clostridium butyricum, Clostridium acetobutylicum, Clostridium saccharoperbutylacetonicum, Clostridium beijerickii, Clostridium saccharobutylicum, Clostridium pasteurianum, Clostridium kluyveri, Clostridium carboxidovorans, Clostridium phytofermentens, Clostridium thermocellum, Clostridium cellulolyticum, Clostridium cellulovorans, Clostridium clariflavum, Clostridium ljungdahlii, Clostridium acidurici, Clostridium tyrobutyricum, and Clostridium autoethanogenum.

According to an embodiment, said method further comprises adding a basic compound for pH control, so that the pH of the fermentation liquor is about neutral, e.g. greater than 5, greater than 5.5 or greater than 6. According to an embodiment, said carboxylic acid RCOOH is at least partially dissociated (in anionic form) in the fermentation liquor, which could be referred to as comprising the salt of the acid. According to an embodiment, said basic compound is calcium hydroxide and/or calcium carbonate, so that the fermentation broth comprises the calcium salt of the carboxylic acid. According to an embodiment, said carboxylic acid is butyric acid and said fermentation liquor comprises calcium butyrate.

According to an embodiment, said carboxylic acid is separated from the fermentation broth at said about neutral pH, i.e. in anionic form. According to an embodiment, said separation of the acid in anionic form comprises anion exchange, e.g. adsorbing the acid on an anion exchanger. According to an embodiment, said separation of the acid in anionic form comprises adsorbing the acid on an anion exchanger, followed by desorbing the adsorbed acid. According to an embodiment, said desorbing comprises a thermal treatment, washing with a desorbing acid solution or combination thereof. According to an embodiment, said desorbing acid solution comprises acid and said auxiliary alkanol ROH.

According to an alternative embodiment, the pH of said fermentation liquor is >5.5 and said separating further comprises acidulating. According to an alternative embodiment, said acidulating comprises adding to said fermentation liquor at least one of a mineral acid and an acidic cation exchanger. According to an alternative embodiment, said acidulating comprises adding to said fermentation liquor sulfuric acid. Alternatively, or additionally, said acidulating comprises adding to said fermentation liquor CO₂, e.g. under super-atmospheric pressure, e.g. pressure greater than 3 atmospheres.

According to an embodiment, the method comprises adding a basic compound for pH control of the fermentation liquor, said basic compound is calcium hydroxide and/or calcium carbonate and said acidulating comprises adding sulfuric acid. According to an embodiment, said carboxylic acid is butyric acid, said fermentation liquor comprises calcium butyrate, said addition of sulfuric acid results in the formation of butyric acid (in acid form) and a gypsum precipitate and the method further comprises separation of said precipitate, e.g. filtering, centrifugation or combinations thereof.

According to an embodiment, the method comprises adding a basic compound for pH control of the fermentation liquor, said basic compound is calcium hydroxide and/or calcium carbonate and said acidulating comprises adding CO₂ under super-atmospheric pressure. According to an embodiment, said carboxylic acid is butyric acid, said fermentation liquor comprises calcium butyrate, said addition of CO₂ results in the formation of butyric acid (in acid form) and a calcium carbonate precipitate and the method further comprises separation of said precipitate, e.g. filtering, centrifugation or combinations thereof.

According to an embodiment, said method further comprises producing carboxylic acid RCOOH in a fermentation process to form a fermentation liquor comprising said carboxylic acid RCOOH and separating said carboxylic acid RCOOH from said fermentation liquor.

According to an embodiment, said separating from said fermentation liquor comprises contacting said fermentation liquor with an extractant comprising separated auxiliary alkanol R′OH to form an extract solution comprising auxiliary alkanol R′OH and carboxylic acid RCOOH and the method further comprises adding said extract solution to said reactor. According to this embodiment, auxiliary alkanol R′OH is used for the extraction of the carboxylic acid to form an extract comprising both, said extract is reacted in said reactor to form said ester, said ester is reacted to form said product alkanol RCH₂OH and said auxiliary alkanol R′OH and said auxiliary alkanol R′OH is separated and reused in extraction.

According to an embodiment, said carboxylic acid RCOOH is butyric acid and said auxiliary alkanol R′OH is selected from the group consisting of alkanols comprising between 4 and 18 carbon atoms. According to an embodiment, said carboxylic acid RCOOH is butyric acid and said auxiliary alkanol R′OH is selected from the group consisting of butanols, pentanols, hexanols and combinations thereof. According to an embodiment, said carboxylic acid RCOOH is butyric acid and said auxiliary alkanol R′OH is a butanol or a mixture of butanols.

According to an embodiment, said extractant further comprises a modifier, said extract solution comprises said auxiliary alkanol R′OH, said carboxylic acid RCOOH and said modifier, and the method further comprises removing at least a fraction of said modifier from the extract solution prior to or concurrently with said adding of said extract solution to said reactor, e.g. by distillation. According to an embodiment, said modifier comprises a saturated or an unsaturated hydrocarbon containing from 3 to 30 carbon atoms. According to an embodiment, said modifier comprises a hydrocarbon producible by oligomerization of alkenes. According to an embodiment, said modifier comprises a hydrocarbon producible by oligomerization of alkenes producible by dehydration of said product alkanol RCH₂OH.

According to an embodiment, said separating from said fermentation liquor comprises contacting said fermentation liquor with an extractant comprising said first and/or second separated ester RCOOR′ to form an extract solution comprising said first and/or second separated ester RCOOR′ and said carboxylic acid RCOOH and the method further comprises adding said extract solution to said reactor. According to an embodiment, said ester RCOOR′ is selected from the group consisting of butyl butyrate, pentyl butyrate, hexyl butyrate and combinations thereof. According to an embodiment, said ester RCOOR′ is butyl butyrate.

According to an embodiment, said extractant further comprises a modifier and said extract solution comprises said ester RCOOR′, said carboxylic acid RCOOH and said modifier, and the method further comprises removing at least a fraction of said modifier from the extract solution prior to or concurrently with said adding of said extract solution to said reactor e.g. by distillation. According to an embodiment, said modifier comprises a saturated or an unsaturated hydrocarbon containing from 3 to 30 carbon atoms. According to an embodiment, said modifier comprises a hydrocarbon producible by oligomerization of alkenes. According to an embodiment, said modifier comprises a hydrocarbon producible by oligomerization of alkenes producible by dehydration of said product alkanol RCH₂OH.

According to an embodiment, said first catalyst comprises a polymeric material containing Lewis and/or Bronsted acid sites. According to an embodiment, said first catalyst comprises a solid acid catalyst supported on a polymeric material or on an inorganic oxide.

According to an embodiment, said second catalyst comprises an inorganic metal oxide support. According to an embodiment, said inorganic metal oxide support comprises at least one transition metal. According to an embodiment, said inorganic metal oxide support comprises at least one transition metal and at least one other metal. According to an embodiment, said inorganic metal oxide comprises aluminum oxide. According to an embodiment, said transition metal comprises copper, e.g. at 10% wt to 40% wt of said support. According to an embodiment, said transition metal further comprises at least one other transition metal taken from Group 4, Group 5, Group 6, Group 7, Group 8, Group 9, and/or Group 10. According to an embodiment, said second catalyst is silanized According to an embodiment, said second catalyst comprises copper-carrying aluminum oxide having a silanized surface.

According to an embodiment, the inorganic support of said second catalyst is selected from the group consisting of γ-alumina, silica, titanium oxide, zinc aluminate and combinations thereof. According to an embodiment, the surface area of said inorganic support is in the range between about 40 and about 500 m²/g (square meters per gram) before silinization. (The composition prior to silanization, comprising said inorganic metal oxide support and optionally transition metals and other metals, is referred to herein as the inorganic composition.) According to an embodiment, after silanization the surface area is between about 30 and about 550 m²/g.

Any silanization method is suitable. According to an embodiment, silanization comprises treating said inorganic composition, e.g. inorganic metal oxide support carrying copper and/or other transition metals, with organosilane solution in a solvent, followed by calcination. According to an embodiment, said organosilane is selected from the group consisting of trimethylsilyl acetate, bis(trimethylsilyl) ether, diphenyldiethoxysilane, tri(trimethylsilyl)phosphate, diphenyldiethoxysilane, tris(trimethylsilyl)phosphate and combinations thereof. According to an embodiment, said solvent is selected from the group consisting of ethanol, methanol, ethylene glycol, propanediol, propanol, iso-butanol, water and mixtures thereof. According to an embodiment, calcining comprises maintaining at a temperature in the range of between about 100° C. and about 500° C. for a time between about 1 hour and about 10 hours. According to an embodiment, the silicon content of the silanized composition is between about 0.01 wt % and about 5 wt % of the inorganic metal support.

According to various embodiments, said first catalyst and said second catalyst are dispersed in the reactor in a random manner, added in layers, or contained in defined compartments, e.g. trays.

According to an embodiment, both said first catalyst and said second catalyst comprise an active moiety supported on a support and the active moiety of the first catalyst and the active moiety of the second catalyst are commonly supported on the same support.

According to an embodiment, the reactor is maintained at a pressure between 1 psig and 1000 psig. According to an embodiment, said reactor is maintained at a temperature between about 50° C. and 300° C. According to an embodiment the reaction vessel has different temperatures along the length of the vessel.

According to an embodiment, the WHSV for the feed is from 0.1 to 15, or 0.4 to 10 based on the carboxylic acid content.

According to an embodiment, said reactor further comprises water at a weight fraction between 0.1% and 20%.

According to an embodiment, the molar yield of converting carboxylic acid RCOOH into product alkanol RCH₂OH is greater than 90%, greater than 92%, greater than 94%, greater than 96%, greater than 97% or greater than 98%. According to an embodiment, said carboxylic acid is butyric acid, said product alkanol is butanol and the molar yield of converting butyric acid to butanol is greater than 90%, greater than 92%, greater than 94%, greater than 96%, greater than 97% or greater than 98%.

According to an embodiment, the method further comprises separating said product alkanol RCH₂OH and said auxiliary alkanol R′OH from said mixture to form separated product alkanol RCH₂OH, separated auxiliary alkanol R′OH and optionally a second separated ester RCOOR′. According to an embodiment, said separating comprises distillation.

According to an embodiment, the number of carbon atoms in R is that in R′ minus one. In that case, the number of carbons in the product alkanol RCH₂OH is identical to that in said auxiliary alkanol R′OH. According to an embodiment, the product alkanol RCH₂OH is identical to the auxiliary alkanol R′OH. According to an embodiment, the product alkanol RCH₂OH and the auxiliary alkanol R′OH are both butanols. According to an embodiment, the product alkanol RCH₂OH and the auxiliary alkanol R′OH are both n-butanol.

According to an embodiment, the product alkanol RCH₂OH is identical to the auxiliary alkanol R′OH and said separating said product alkanol RCH₂OH and said auxiliary alkanol R′OH from said mixture forms a stream comprising said separated alkanol. According to an embodiment, said stream comprising said separated alkanol is split into a first fraction forming said separated product alkanol, a second fraction forming said auxiliary alkanol for reuse in the reactor and optionally other fractions.

According to an embodiment, the method further comprises purification and/or drying said separated product alkanol RCH₂OH. According to an embodiment, the method further comprises distillation of the product alkanol RCH₂OH. According to an embodiment, the method further comprises treatment of the product alkanol RCH₂OH on a molecular sieve.

According to an embodiment, the method further comprises dehydrating said separated product alkanol RCH₂OH on a third catalyst, also referred to herein as the dehydrating catalyst, in a reactor. According to an embodiment, said third catalyst comprises an inorganic metal oxide support, optionally modified with a promoter. According to an embodiment, said inorganic support comprises γ-alumina, for example, in the form of a rod-like extrudate, silica, titanium oxide, zinc aluminate, or combinations thereof. According to an embodiment, said third catalyst is not modified with a promoter. According to an embodiment, said third catalyst is silanized According to an embodiment, said third catalyst is silanized by treatment with an organosilane in a solvent, followed by calcination, as in said second catalyst. According to an embodiment, said catalyst is a silanized, surface-stable selective dehydration catalyst for internal alkene production, comprising (i) an inorganic support, and (ii) at least one silicon compound.

According to an embodiment, the method further comprises chemically catalyzed dehydrating said product alkanol to a corresponding alkene (on said third catalyst) and optionally chemically catalyzed oligomerizing said alkene on a fourth catalyst, also referred to herein as the oligomerization catalyst, in a reactor. According to an embodiment, said product alkanol is n-butanol and the product of oligomerization is a fuel, e.g. hydrocarbon jet fuels (ASTM D7655). Said reaction comprising dehydrating and oligomerizing is shown schematically below for the case where the product alkanol is butanol (catalyst A and catalyst B are the third catalyst and the fourth catalyst, respectively).

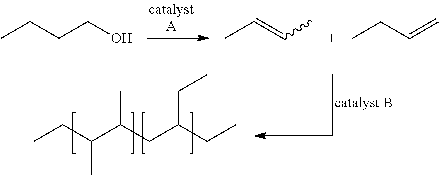

In some embodiments, the alkene is produced with about 92% to about 99% chemical yield. In some embodiments, a single pass over the dehydrating catalyst affords a chemical conversion of greater than about 94%, or about 97%, or about 99.5%. In some embodiments, the alkene is produced employing WHSV values greater than 1.0 hr⁻¹, e.g., greater than 1 and less than 15 hr⁻¹, or about 1.5 hr⁻¹ to about 8 hr⁻¹. In some embodiments the dehydrating reactor is maintained at a temperature of about 200° C. to about 440° C., or about 250° C. to about 370° C. and at a pressure of about 2 psig to about 100 psig. In one embodiment, n-butanol formed according to the method of the present invention from butyric acid is dehydrated to a mixture of butenes in a chemical yield of greater than about 95%, or greater than 97%, or greater than 99% yield.

In one embodiment, the dehydration catalyst is prepared by treating at least one inorganic oxide with at least one organosilane. According to an embodiment, this treating is done using incipient wetness techniques. Alternatively, the organosilane is reacted with the inorganic oxide by contacting organosilane vapors with the solid at temperatures ranging from ambient to 300° C.

In some embodiments, the dehydration catalyst is modified by treatment with at least one organosilane and promoter dissolved in an alcohol solvent. In one embodiment, the organosilane is diphenyldiethoxysilane and alcohol solvent is ethanol. In another embodiment, the organosilane is diphenyldiethoxysilane and alcohol solvent is methanol containing 5 wt-% water. The alcohol solvent can contain water from about 10 ppm up to about 95 wt-%. For selected organosilane modifiers that possess adequate water solubility (i.e. greater than 0.5 wt-%), water alone can be used to deliver the organosilane and acid to the catalyst surface.

In some embodiments of the methods, at least one purge gas is provided, e.g., nitrogen and/or argon. In other embodiments the inert gas/purge gas (e.g. nitrogen) can have at least one hydrocarbon (e.g. butane) added from 1 to 99 wt-% as a function of the alcohol feed to the dehydration reactor.

The silanized dehydration catalyst formed thereby is found to be more reactive and to have an unexpected propensity for creating the internal alkene isomers, compared to non-silanized catalysts known in the art. The invention catalysts have a unique ability to create a non-equilibrium ratio of 1-alkene/internal-alkene when dehydrating terminal alcohols. One example is that the dehydration of 1-butanol affords the 2-butenes in greater than 90% selectivity and at a significantly faster rate when compared to typical promoted dehydration catalysts (e.g. sodium doped gamma-alumina) known in the art.

In some embodiments, the dehydration catalyst can be used continuously for at least 1 to 3 months, or about 6 months and in some embodiments, up to about 12 months, or up to about 18 months, or periods of greater than 20 months while producing butenes without significant loss in chemical selectivity for the internal alkene nor a decrease in rate of reaction.

According to an embodiment, said dehydrating said product alkanol is conducted in at least one isothermal continuous flow reactor. In one embodiment, a series of one or more adiabatic reactors is used to dehydrate an alcohol or diol to at least one internal alkene product. In some embodiments, some of the heat necessary for the chemical dehydration reaction is carried into the reactor in the form of a gaseous diluent, such as steam or a mixture of at least one hydrocarbon or at least one inert gas. In the case of water addition, this can generate a very water rich gas phase in the reactor making some embodiments of the methods disclosed herein beneficial for commercial applications.

According to an embodiment, the method further comprises chemically-catalyzed oligomerization of the formed butene mixture. In some embodiments, said oligomerization is conducted on a mesoporous oligomerization catalyst. In various embodiments, the oligomers may be used to produce a diesel fuel (e.g., with a flashpoint of about 38 to about 100° C., a Cetane rating of about 40 to about 60, and aromatic content of less than about 0.5 wt-%), a jet fuel (e.g., with a flashpoint of about 38 to about 100° C., a cold flow viscosity of less than about 8.0 cSt (centistokes) at −20° C., and aromatic content of less than about 0.5 wt-%), or a lubricant (e.g., with a viscosity of about 1 to about 10,000 cSt at 25° C.).

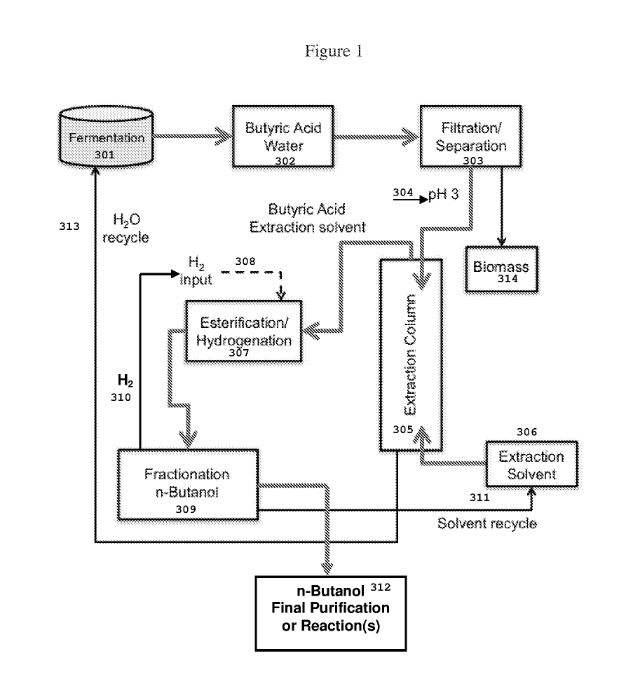

An embodiment of the method is presented in FIG. 1. The fermentation liquor comprising among other things butyric acid and water (302) produced in the fermentation process (301) is filtered (303) to remove solids (biomass, 314) therefrom. The pH of the filtered fermentation liquor is adjusted to 3 by means of sulfuric acid (304). The acidulated fermentation liquor is sent to an extraction column (305) where it is extracted by butanol (the extraction solvent) (306). The extraction solvent containing the butyric acid and butanol (the extract) is sent to a reactor (307) comprising said first catalyst and said second catalyst, wherein esterification and hydrogenation (hydrogen input shown at 308) take place. The reaction product is sent to separation/fractionation (309), forming butanol for recycle to extraction (311), optionally excess H₂ for recycle to esterification/hydrogenation (310), and product butanol for final purification and/or drying and optionally further reaction (312). A fraction of the raffinate (the carboxylic acid-depleted aqueous solution) is optionally recycled to the fermentation reaction (313).

According to an alternative embodiment, the acidulated fermentation liquor is extracted by butyl butyrate to form an extract containing the butyric acid and butyl butyrate. A fraction of the ester is separated from the extract and recycled back to extraction. The ester-depleted extract is then sent, along with butanol, to a reactor comprising said first catalyst and said second catalyst, wherein esterification and hydrogenation take place.

According to an embodiment, butyric acid is dimerized to form 4-heptanone (a reaction commonly known as ketonization). According to an embodiment, in this process, one mole of carbon dioxide is produced for each mole 4-heptanone:

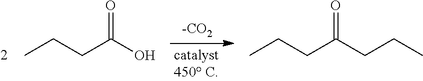

According to an embodiment, the method further comprises converting said 4-heptanone to 4-heptanol, dehydrating to heptenes, selectively dimerizing, and finally hydrogenating, whereby a fourteen-carbon saturated hydrocarbon is formed. According to an embodiment, the method further comprises blending said fourteen-carbon saturated hydrocarbon with jet or diesel fuel.

What is claimed is:

1. A method for the production of at least one derivate of a carboxylic acid RCOOH, comprising
(i) reacting in a reactor a carboxylic acid RCOOH with an auxiliary alkanol R′OH on a first catalyst to form a produced ester RCOOR′;
(ii) optionally, separating a fraction of said produced ester RCOOR′ to form a first separated ester RCOOR′;
(iii) reacting in said reactor at least a fraction of said produced ester RCOOR′ with hydrogen on a second catalyst to produce a mixture comprising a product alkanol RCH₂OH and said auxiliary alkanol R′OH and optionally residual ester RCOOR′;
(iv) separating said product alkanol RCH₂OH and said auxiliary alkanol R′OH from said mixture to form separated product alkanol RCH₂OH, separated auxiliary alkanol R′OH and optionally a second separated ester RCOOR′; and
(v) recycling said separated auxiliary alkanol R′OH to said reactor;
wherein
R and R′ denote alkyl and/or aryl groups, and
steps (i) and (iii) are conducted in said reactor concurrently.

2. A method according to claim 1, wherein R in said carboxylic acid RCOOH comprises 1 to 30 carbon atoms.

3. A method according to claim 1, wherein said carboxylic acid RCOOH is butyric acid and said product alkanol RCH₂OH is butanol.

4. A method according to claim 1, wherein R′ in said auxiliary alkanol R′OH comprises 1 to 30 carbon atoms.

5. A method according to claim 1, further comprising producing a carboxylic acid RCOOH and obtaining a solution comprising said carboxylic acid RCOOH and said auxiliary alkanol R′OH, wherein said separated auxiliary alkanol R′OH is present during at least part of said carboxylic acid RCOOH-producing step.

6. A method according to claim 1, further comprising producing a carboxylic acid RCOOH and obtaining a solution comprising said carboxylic acid RCOOH and at least one separated ester RCOOR′, wherein at least one of said first separated ester RCOOR′ and said second separated ester RCOOR′ are present during at least part of said carboxylic acid RCOOH-producing step.

7. A method according to claim 1, further comprising producing carboxylic acid RCOOH in a fermentation process to form a fermentation liquor comprising said carboxylic acid RCOOH and separating said carboxylic acid RCOOH from said fermentation liquor.

8. A method according to claim 7, wherein said separating from said fermentation liquor comprises contacting said fermentation liquor with an extractant comprising separated auxiliary alkanol R′OH to form an extract solution comprising auxiliary alkanol R′OH and carboxylic acid RCOOH, and wherein the method further comprises adding said extract solution to said reactor.

9. A method according to claim 8, wherein said extractant further comprises a modifier and wherein said extract solution comprises an auxiliary alkanol R′OH, a carboxylic acid RCOOH and said modifier, and the method further comprises removing at least a fraction of said modifier from the extract solution prior to or concurrently with said adding to said reactor.

10. A method according to claim 9, wherein said modifier comprises a saturated or an unsaturated hydrocarbon containing from 3 to 30 carbon atoms.

11. A method according to claim 7, wherein said separating from said fermentation liquor comprises contacting said fermentation liquor with an extractant comprising a first and/or second separated ester RCOOR′ to form an extract solution comprising said first and/or second separated ester RCOOR′ and said carboxylic acid RCOOH, and wherein the method further comprises adding said extract solution to said reactor.

12. A method according to claim 11, wherein said extractant further comprises a modifier and said extract solution comprises said ester RCOOR′, said carboxylic acid RCOOH and said modifier, and the method further comprises removing at least a fraction of said modifier from the extract solution prior to or concurrently with said adding of said extract solution to said reactor.

13. A method according to claim 12, wherein said modifier comprises a saturated or an unsaturated hydrocarbon containing from 3 to 30 carbon atoms.

14. A method according to claim 7, wherein the pH of said fermentation liquor is >5.5 and said separating further comprises acidulating with at least one of a mineral acid and an acidic cation exchanger.

15. A method according to claim 14, comprising providing a fermentation medium comprising a carbon source, culturing in said liquor a carboxylic acid RCOOH-producing organism and adding a basic compound for pH control.

16. A method according to claim 15, wherein said basic compound is calcium hydroxide and/or calcium carbonate and said mineral acid is sulfuric acid.

17. A method according to claim 15, wherein said carbon source is selected from the group consisting of sugars, glycerol, methanol, CO, CO2, syngas and combinations thereof.

18. A method according to claim 15, wherein said organism is one or more of: a member of the phylum Firmicutes, a member of the class Clostridia, a member of the genus Eubacterium, a Eubacterium limosum, and a Clostridium selected from Clostridium butyricum, Clostridium acetobutylicum, Clostridium saccharoperbutylacetonicum, Clostridium beijerickii, Clostridium saccharobutylicum, Clostridium pasteurianum, Clostridium kluyveri, Clostridium carboxidovorans, Clostridium phytofermentens, Clostridium thermocellum, Clostridium cellulolyticum, Clostridium cellulovorans, Clostridium clariflavum, Clostridium ljungdahlii, Clostridium acidurici, Clostridium tyrobutyricum, and Clostridium autoethanogenum.

19. A method according to claim 1, wherein said first catalyst comprises a polymeric material containing Lewis and/or Bronsted acid sites.

20. A method according to claim 1, wherein said second catalyst comprises aluminum oxide containing from 10 to 40 weight percent copper.

21. A method according to claim 1, wherein said second catalyst is silanized.

22. A method according to claim 1, wherein said second catalyst comprises copper-containing aluminum oxide having a silanized surface.

23. A method according to claim 1, wherein said second catalyst comprises an inorganic oxide, a transition metal, and a silanized surface.

24. A method according to claim 1, wherein both said first catalyst and said second catalyst comprise an active moiety supported on a support and wherein the active moiety of the first catalyst and the active moiety of the second catalyst are commonly supported on the same support.

25. A method according to claim 1, wherein said reactor is maintained at a temperature between about 50° C. and 300° C.

26. A method according to claim 1, wherein said reactor is maintained at a pressure between 1 psig and 1000 psig.

27. A method according to claim 1, wherein said reactor further comprises water at a weight fraction between 0.1% and 20%.

28. A method according to claim 1, wherein the molar yield of converting carboxylic acid RCOOH into product alkanol RCH₂OH is greater than 90%.

29. A method according to claim 1, further comprising dehydrating said separated product alkanol RCH₂OH on a third catalyst.

30. A method according to claim 29, wherein said third catalyst comprises a silanized inorganic metal oxide.

31. A method according to claim 29, wherein said catalyst is a silanized, surface-stable selective dehydration catalyst for internal alkene production, comprising (i) an inorganic support, and (ii) at least one silicon compound.
